(19) United States
(12) Patent Application Publication
(10) Pub. No.: US 2020/0352744 A1
(43) Pub. Date: Nov. 12, 2020

(54) SYSTEM MADE UP OF MULTIPLE ORTHOPEDIC COMPONENTS AND METHOD FOR CONTROLLING SUCH A SYSTEM

(71) Applicant: Otto Bock Healthcare Products GmbH, Wien (AT)

(72) Inventors: Robert KAITAN, Wien (AT); Andreas WÜSTEFELD, Obernfeld (DE); Alexander PAPPE, Wien (AT); Robert HOFFMANN, Wien (AT); Karl-Heinz KLINGEBIEL, Duderstadt (DE); Thomas PAUSER, Steinbrunn (AT); Erik ALBRECHT-LAATSCH, Rosdorf (DE); Lüder MOSLER, Duderstadt (DE); Michael NOLTE, Seeburg (DE); Luis SAGMEISTER, Pitten (AT); Andreas WEIGL-POLLACK, Goldgeben (AT); Andreas SCHRAMEL, Wien (AT)

(73) Assignee: Otto Bock Healthcare Products GmbH, Wien (AT)

(21) Appl. No.: 15/733,269

(22) PCT Filed: Dec. 18, 2018
(86) PCT No.: PCT/EP2018/085665

§ 371 Date: Jun. 18, 2020

(30) Foreign Application Priority Data
Dec. 22, 2017 (DE) 10 2017 131 190.4

(51) Int. Cl. A61F 2/60; A61F 2/70

(52) U.S. Cl. CPC A61F 2/60; A61F 2002/5083; A61F 2/70

(57) ABSTRACT

A system made up of multiple orthopedic components which are coupled to one another, having a first electronic and/or electric device which has a first supply terminal via which the first electric and/or electronic device can be supplied with energy and/or data from a charging station via a plug. At least a second electric and/or electronic device is provided on one of the components which has a separate, second supply terminal and/or a plug connection which can be coupled to the first electronic and/or electric device for transmitting energy and/or data via the first supply terminal or a separate plug connection.

The invention relates to a system made up of multiple orthopedic components which are coupled to one another, having a first electronic and/or electrical unit, which comprises a first supply terminal, via which the first electrical and/or electronic unit can be supplied with energy and/or data from a charging station via a plug. The invention also relates to a method for controlling such a system.

Orthotics, prostheses, wheelchairs, data loggers, radio modules, feedback elements, electrical storage units, or parts of orthotics or prostheses or wheelchairs are considered to be orthopedic components, for example, prosthetic joints, prosthetic feet, tubular adapters, prosthetic hands, prosthetic elbows, rotating adapters, prosthesis sockets, orthotic rails, orthotic joints, foot rests, and sensors, storage units, processors, or other data processing units arranged thereon. A system made up of multiple orthopedic components is a combination of such components to form an orthopedic unit, which has a functionality going beyond the functionality of the individual orthopedic component. Such a system can be, for example, a femoral prosthetic made of a femoral socket, a prosthetic knee joint, a transtibial tube, and a prosthetic foot. The system of a transtibial prosthesis consists of a transtibial socket and a prosthetic foot arranged thereon. Prostheses of an upper extremity as an upper arm prosthesis can comprise a socket, input devices, controt devices, power supplies such as batteries, rechargeable batteries, capacitors, transformers, or power supply units, a shoulder, an active elbow, a wrist, gripping devices, for example, a gripper and/or individual fingers. An orthotic system can be formed, for example, as a knee-ankle-foot orthotic (KAFO).

Orthopedic components are frequently provided with electrical and/or electronic units, for example, to detect active forces, spatial positions, torques, or assignments of components to one another. In addition, a change of resistances of a damper unit or an activation or deactivation of a motorized drive can be performed on the basis of ascertained sensor values, which are analyzed in a processor. The useful data of the orthopedic components can be analyzed, for example, to have a data foundation for future settings. In addition, function tests can be carried out by connected testing units. Complete prosthetic or orthotic units, such as computercontrolled arm prostheses or exoskeletons, are required for complex treatments.

In particular in more complex orthopedic systems, every actuator or electrical consumer comprises an energy accumulator assigned to it. In addition, the adaptations, for example, of a prosthetic knee joint with an actuatable prosthetic ankle joint can be complex and possibly can be carried out independently of one another. Electronic units can also be provided in non-driven components, for example, to detect loads or angular positions via integrated sensors. Due to the modular construction and prosthetics or orthotics, a variety of different possible combinations exist, which can increase the complexity in the case of a software adjustment or an energy supply.

The object of the present invention is therefore to provide a system made up of multiple orthopedic components and a method for controlling such a system, which is easier to handle and enables a uniform supply with data and/or electric energy.

This object is achieved according to the invention by a system having the features of the main claim and a method having the features of the concurrent claim. Advantageous designs and refinements of the invention are disclosed in the dependent claims, the description, and the figures.

The system according to the invention made up of multiple orthopedic components which are coupled to one another, having a first electronic and/or electrical unit, which comprises a first supply terminal, via which the first electrical and/or electronic unit can be supplied with energy and/or data from a charging station via a plug, provides that at least one second electrical and/or electronic unit is arranged on one of the components, which comprises a separate second supply terminal and/or a plug terminal, which can be coupled to the first electronic and/or electrical unit to transmit energy and/or data via the first supply terminal or a separate plug terminal. The two components can comprise different terminals for connection to a charging station and for connection to one another. The requirements for a connection of one supply terminal to a charging station can differ from the requirements for a connection between two components, which frequently have a long-term connection to one another. The frequency of making contact and breaking contact differs, as do the requirements for a touch protection or a water-tightness, so that the plugs for supply terminals can be designed differently than the plugs for plug terminals or plug sockets. In principle, plug terminals and supply terminals can be compatible with one another, so that using one plug or one plug type, a connection can be established both to a charging station and also between two components, wherein not all functions or configurations have to be activated for different connections. The plug terminal can have a higher IP protection class than the supply terminal, since the supply terminal is generally potential-free in the case of an unplugged contact. It is possible via a separation of supply terminal and plug terminal to take into consideration the different requirements for an energy transmission alone, which is frequently short, having frequent contacting, and a permanent plug connection having energy and data transport.

One refinement of the invention provides that the first and second electronic and/or electrical unit can be coupled to one another via a plug connection between the plug terminals, the supply terminals, or one plug terminal and one supply terminal, whereby the option exists of designing all plugs uniformly. Alternatively, in the case of different functions, in particular if they preclude one another, different plug shapes can be formed to avoid inadvertent incorrect contacting.

The supply terminals of the first and second electronic and/or electrical units can be compatible with the plug terminal. Via the second supply terminal or a plug terminal which is compatible with the plug to supply the first electrical and/or electronic unit, it is possible to provide a simple communication between the at least two orthopedic components within the system. In addition, it is possible to supply the respective orthopedic components and the electrical and/or electronic units arranged therein or thereon with energy, in particular to charge the electrical energy accumulators or rechargeable batteries and in addition to read out and analyze the data acquired during the operation. It is moreover possible, using only one charging station, via the compatible design of the supply terminals and possibly plug terminals to supply all electrical and/or electronic units using one charging station, via which the energy and/or data are fed. The two or more orthopedic components are thus considered to be one unit, which can be centrally supplied and can be handled in a manner matching with respect to its special functionality, which is adapted to the respective patient.

The electrical and/or electronic unit can comprise an actuator, for example, a motorized drive, a hydraulic drive or damper, an interlock, or a pneumatic unit, and/or a control unit, a processing circuit or processor, a sensor, a data memory, a charging unit, and/or an energy accumulator. The charging device or the charging unit can be designed as one of the communicating devices and can participate in the bus system via which all components of the system communicate with one another.

The orthopedic components are preferably mechanically and/or electrically coupled to one another. It is possible via the mechanical coupling of the components to one another to enable pivot movements in the case of an articulated connection and to enable other displacements of the components in relation to one another in the case of other guide units. In addition, forces or torques can be transmitted, for example, to be able to absorb ground reaction forces when walking and conduct them through onto the body. Forces and torques can be transmitted from one component to the other. It is possible via the mechanical coupling, in the case of a modular construction of orthopedic components, such as orthotics, prostheses, or wheelchairs, to assemble an orthotic, prosthesis, or another orthopedic unit adapted to the respective patient from many individual parts.

An electrical coupling to the charging station or between two supply terminals can be implemented, for example, via a plug connection, in which at least one plug side is a cascading plug, which comprises a plug formation corresponding to the side facing toward the supply terminal and forms a receptacle or socket for the plug, as it comes from the charging station, on the side facing away. The wiring can extend within the orthopedic system, for example, the prosthesis or the orthotic, to protect the cables from external influences. The plugs can also be arranged inside the orthopedic system, wherein the accessibility from the outside can be provided, for example, in the case of attached prosthesis or orthotic. At least one cable branches off from this plug constructed in a cascading manner to a plug which is attachable to the second supply terminal. Via such a construction, two or more orthopedic components having supply terminals can be electrically and electronically coupled to one another to introduce or transmit data and/or energy.

Exclusively data, exclusively electrical energy, or both can be transmitted via the second supply terminal, so that the second electrical and/or electronic unit can be supplied with the required items of information and energy.

One refinement of the invention provides that an adapter is designed to be connectable to the first or second supply terminal or plug terminal and the adapter comprises an input terminal for different charging stations. The option exists via such an adapter of using different energy sources, for example, to supply energy accumulators in the orthopedic component. Energy sources having different voltage supply can be connected to the orthopedic component via the adapter in order to ensure a supply with energy substantially independently of the location. Via the adapter, connections can be established to 12 V supplies, for example, via a cigarette lighter terminal, to 5 V terminals, to USB terminals, to terminals of battery packs, or to a computer or a notebook. Different voltage levels can be processed and relayed on the data lines and/or energy lines via the adapter. It is very generally possible via the adapter to use different communication protocols.

One refinement of the invention provides that the supply terminal and/or plug terminal comprises a fastening unit for fixation on the respective electrical and/or electronic component. The fastening unit can be formed as a formfitting fixation and/or friction-locked unit. It is thus possible that the supply terminal as such can be fixed variably or also only individually positionable on the component. Formfitting fastening units can be formed as hook and loop fasteners, clips, or threads. A threaded rod or a threaded bushing can be arranged on the supply terminal, which is then fixable in a formfitting manner on the component via a screw connection using a corresponding nut or screw. Alternatively or additionally, the supply terminal can be positioned, preferably reversibly, at a point of the component advantageous for the patient or orthopedic technician via a hook and loop fastener, an adhesive layer, or also a magnet. A cable connection is preferably provided between the supply terminal and the actual electrical or electronic unit, which is laid in or on the component and is possibly shielded via a cover from external influences.

A further design of the invention provides that a control or distribution unit having multiple electrical and/or electronic components is coupled to accumulators of electric energy, wherein the control or distribution unit detects the charge levels of the respective accumulators of electric energy and, in dependence on the respective charge levels of the accumulators, distributes a charging current from the supply terminal to the accumulators or distributes electric energy among the accumulators, so that the most uniform possible utilization of the storage capacity of the electrical accumulators is performed. It is thus possible that an energy exchange takes place between the components and the individual electrical or electronic units or a corresponding amount of energy or charging current is assigned by the control or distribution unit to the respective accumulator in dependence on the respective charge level of the accumulator. If the energy accumulator is empty or has the lowest charge level in comparison to the other energy accumulators, this energy accumulator is preferably provided with charging current, since it is to be assumed that the electrical or electronic units assigned to this accumulator are used particularly frequently or have a high energy consumption and therefore preferably have to be supplied with additional energy. It is also possible via the control or distribution unit to execute an energy balancing within the orthopedic components or within the system, so that the capacity of all energy accumulators in the system of the orthopedic components can be utilized as uniformly as possible. For this purpose, differences in the charge levels of the respective accumulators are analyzed and in the case of a high charge level, the energy is distributed to an accumulator having a lower charge level.

The electrical and/or electronic units are coupled to one another electrically and/or to transmit data, in order to reach all electrical and/or electronic units both with respect to the energy supply and also with respect to a data exchange or a data reconciliation via a central terminal. In addition, an identification or an identifiability of the individual electrical and/or electronic units is produced via the electrical and/or electronic coupling, so that an individually assembled orthopedic system, for example, an orthotic or prosthesis, having multiple components can be detected in its entirety by the respective charging station and can be individually addressed with respect to the charging procedure and/or the supply with data.

In one refinement of the invention, all electrical and/or electronic units are connected to a control unit, which identifies all electrical and/or electronic units with respect to the function, the construction, and the type thereof and prompts the activation and deactivation thereof from the control unit. Due to the automatic identification by the control unit, the charging station is supplied with all required items of information with respect to the quantity of energy to be provided and the type of the scope of the data to be provided or also the query modalities of the data recorded within the orthopedic component.

The method according to the invention for controlling an above-described system provides that all electrical and/or electronic units provided in the system and coupled to one another are triggered by one control unit. Every electrical and/or electronic unit is addressed or moved to a reaction via a data query by the triggering, so that the status and the functional scope of the respective electrical and/or electronic unit is queried. After the query, energy and/or data signals are transmitted to the respective electrical and/or electronic unit, so that a data and energy supply adapted to the respective component or customized thereto can take place.

One refinement of the method provides that the charge levels of multiple electric energy accumulators are detected and the charging energy is distributed on the basis of the detected charge levels during a charging procedure. In particular, energy accumulators having a high charge capacity and/or energy accumulators having a low charge level are preferably supplied with electric energy. The preferred or particularly important or large accumulators can be supplied over a longer period of time or with a higher charging current to be able to supply the entire system with the largest possible quantity of energy.

Multiple electric energy accumulators can be combined to form a composite, so that all energy accumulators can supply all electrical and/or electronic units with energy. The supply of the electrical and/or electronic units from the composite is distributed centrally by the control unit, so that it is ensured that all electrical and/or electronic components can be supplied over a maximum run time with a sufficient quantity of electric energy. In addition, a prioritization of the respective electrical and/or electronic components can be performed. If some electrical and/or electronic components are not essential for the functionality of the orthopedic system, in the case of a critically low energy level, they can be switched off or reduced in the functional scope thereof.

One refinement provides that the electrical energy accumulator which has the highest charge level is first assigned to a consumer. This assignment can be performed independently of the position assignment of the respective energy accumulator to an orthopedic component of the system. For example, an energy accumulator on a femoral rail, which is actually provided for supplying a drive of an orthotic knee joint, can be used to actuate an orthotic ankle joint if the energy accumulator on the femoral rail has a larger quantity of energy available than the energy accumulator actually assigned to the ankle drive.

The energy accumulators can carry out a charge exchange with one another to ensure a uniform utilization of the energy accumulators.

The function of the electrical and/or electronic units can be individually activated or deactivated in each case due to the coupled components. Each component has a functional scope which is assigned to the respective component and can be queried by the control unit. Several functions can be counterproductive or are not required in combination with other orthopedic components, so that an automatic switching off or activation of individual functions can be performed in dependence on the functional scope and the indication resulting from the combination of the individual orthopedic components.

One refinement of the invention provides that the electrical and/or electronic units are authenticated after the coupling of the orthopedic components, and the functional scope of the electrical and/or electronic units and/or an energy release to these units is established in dependence on the electrical and/or electronic units combined with one another. Such an authentication prevents functions from being enabled which would be harmful for the overall system.

Exemplary embodiments of the invention are explained in greater detail hereafter on the basis of the appended figures. In the figures:

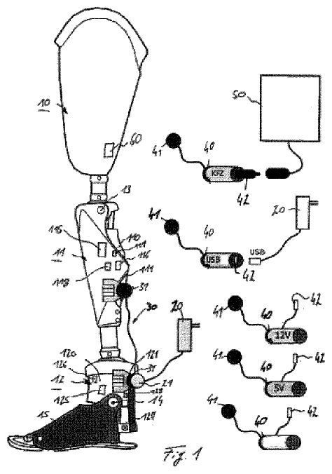

FIG. 1—shows a first variant of the system; and

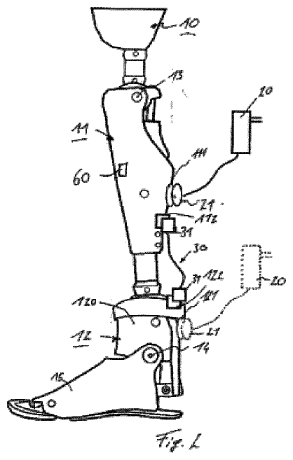

FIG. 2—shows a second variant.

A prosthesis of a lower extremity having multiple orthopedic components 10, 11, 12, 15 is shown in a schematic illustration in FIG. 1. A femoral socket having a connecting adapter for fastening on an upper part of a prosthetic knee joint is shown as a first orthopedic component 10. A second orthopedic component 11 in the form of a transtibial part is arranged on the prosthetic knee joint so it is pivotable around a pivot axis 13. A third orthopedic component 12 in the form of a prosthetic ankle joint is fastened on the transtibial part as the second orthopedic component 11, on which ankle joint a prosthetic foot 15 is mounted so it is pivotable around a pivot axis 14 as the fourth orthopedic component. The femoral socket as the first orthopedic component 10 is a solely passive component and is used to accommodate a femoral stump, to be able to fix the prosthetic leg securely on the patient. Via the femoral socket and a prosthetic liner arranged therein, for example, a mechanical fixation of the prosthetic leg on the patient can be performed via a partial vacuum system. Alternatively to a multipart prosthesis, the system can be formed as an orthotic, a wheelchair or a prosthetic or orthotic unit for an upper extremity can also be used as an orthopedic component, for example, a prosthetic arm, an arm orthotic, or a shoulder orthotic. Combinations of prosthetics, orthotics, and wheelchairs or other mobility aids are also considered to be a system.

In the illustrated system, a first electrical and/or electronic unit 110 in the form of a hydraulic actuator is arranged in the second orthopedic component 11. The hydraulic actuator can be formed as a passive component and can comprise a hydraulic damper, which comprises positioning drives, via which valves are opened or closed to be able to set an extension resistance and/or flexion resistance. Moreover, an accumulator 115 for storing electric energy can be arranged on the hydraulic actuator or assigned thereto in order to supply the positioning drives with energy. In a design of the electrical and/or electronic unit 110 as an active actuator, a pump unit or a mechanical energy accumulator, for example, a spring or pressure accumulator, is assigned to the hydraulic damper, via which it is possible to effectuate or assist a movement of the femoral socket in relation to the lower leg. For this purpose, the energy from the energy accumulator 115 is converted into movement energy, so that, for example, a piston rod is moved out of the hydraulic actuator to assist or effectuate an extension movement. Vice versa, hydraulic fluid can be circulated by a pump or a pressure accumulator, so that a piston rod is retracted into a housing of the hydraulic actuator, in order that the distance between two fastening points of the hydraulic actuator on the upper part and the lower part of the prosthetic joint is shortened in order to execute a flexion movement.

The second electrical and/or electronic component 120 is arranged distal to the first electrical and/or electronic component 110 in the orthopedic component 12 in the form of a prosthetic ankle joint. An electrical and/or hydraulic actuator can also be arranged inside the prosthetic ankle joint, which can be supplied with electric energy via an accumulator 125 for electric energy. In addition to the accumulator 125 for electric energy, a control unit 126 is arranged in the prosthetic ankle joint, the control unit 116 is arranged in the first electrical and/or electronic unit 110, for example, to activate or deactivate a drive 118, 128, or to process data of a sensor 117, 127, to store sensor data, and to use these data further for the control. A data memory and a processing circuit can also be integrated into the control unit 116, 126.

A supply terminal 111, 121 is arranged in each case on both the second orthopedic component 11 and also on the third orthopedic component 12, via which energy and/or data can be supplied to the respective electrical and/or electronic unit 110, 120. The data and the electric energy can be transmitted from a charging station 20 to the respective supply terminal 111, 121. For this purpose, a plug 21, which is compatible with the respective supply terminal 111, 121, is arranged on the charging station 20. In the illustrated exemplary embodiment of FIG. 1, the plug 21 of the charging station 20 is coupled to a cascading plug system of a plug connection 30, which connects the two supply terminals 111, 121 to one another. Two plugs 31 are arranged on the plug connection 30, which comprise contacts on one side, which are formed compatibly with the contacts of the respective supply terminals 111, 121. On the side facing away from the supply terminals 111, 121, the plugs 31 comprise receptacles or sockets, which are compatible with the contacts of the plug 21 of the charging station 20.

If the plug 21 of the charging station 20 is plugged onto the rear side of a plug 31, which is connected via a cable to a corresponding plug 31, so that both supply terminals 111, 121 are connected to one another, energy and data can be transmitted from the charging station 20 both to the first and also to the second electronic and/or electrical unit 110, 120. The accumulators 115, 125 for storing electric energy are thus filled and also the control units 116, 126 are supplied with data such as programs, control data, software updates, or the like. Via the system made up of charging station 20 having plug 21, supply terminals 111, 121, and the plug connection 30 having the plugs 31, it is possible to provide an electrical connection system for orthopedic components 11, 12, in particular orthotics, prostheses, and/or wheelchairs, using which it is possible to charge the respective electrical and/or electronic units 110, 120, connect them to one another to distribute energy from the respective energy accumulators 115, 125 among one another, or to coordinate control sequences with one another. It is possible via the system not only to supply external data from the charging station 20 and electric energy to the orthopedic system, for example, the orthotic, prosthesis, or the wheelchair, but rather also to enable an energy and/or data exchange within the orthopedic unit between the respective orthopedic components 11, 12.

If different maximum required powers are present in the respective consumer, for example, drive 118, 128, or for charging the accumulators 115, 125 in the different components 11, 12 having the various electrical and/or electronic units 110, 120, the respective maximum required power of a consumer 118, 128 or a control unit 116, 126 can be coded via an electrical resistor. It is possible due to the compatibility of the supply terminals 111, 121 with the respective plugs 21, 31 to provide multiple charging terminals on an orthopedic component in order to facilitate coupling the orthopedic aid to a charging station 20 for a patient. The user can thus freely select the supply terminal 111, 121 to be reached best by him, so that one or more terminals are provided for the central charging of all electrical and/or electronic units 110, 120. The charging with electric energy and also the supply with data can take place simultaneously or sequentially. If, for example, the electrical and/or electronic units 110, 120 are not coupled to one another via the plug connection 30, data and energy can be transmitted in succession via the charging station 20 through the plug 21. The respective electronic and/or electrical unit 110, 120 is provided with a code, so that it is recognized via the charging station 20 which unit is presently supposed to be supplied with energy and/or data, so that both the correct quantity of energy and also the correct data are transmitted.

If the charging station 20 is not connected, a data and energy exchange can take place between the units 110, 120 via the plug connection 30. It is also possible to combine multiple orthopedic aids with one another via a plug connection 30, for example, an orthotic or a prosthesis with a wheelchair, which has, for example, a larger energy accumulator in the form of a battery, so that energy can be transmitted from the wheelchair into the orthotic or prosthesis to maintain the mobility of the patient.

Further charging stations 20 and energy accumulators are shown in FIG. 1, which provide different voltages, for example a 12 V voltage, a 5 V voltage, or various voltages via a special transformer or a producer-specific charging device. An energy accumulator can also be coupled via a USB terminal to a power supply unit of a charging station 20, so that a mobile accumulator for electric energy is provided. A terminal for a cigarette lighter can also be used as an energy supply, which can also be coupled to an external energy accumulator 50, for example, on which a corresponding adapter is arranged. The external energy accumulator 50, which can also be a computer or portable computer, can also be coupled to an orthopedic component independently of a cigarette lighter. Via one or more adapters 40, which are provided with a respective plug 41 which are compatible with the respective supply terminals 111, 121, it is possible to use greatly varying energy sources to supply the respective electronic and/or electrical unit with energy. The adapters 40 can connect different types of energy sources to the respective orthopedic components, wherein different voltage levels can be fed to the respective units within the adapter 40. The respective required voltage level can be configured within the adapter 40, for example, by increasing or reducing the voltage received from the respective energy accumulator. An input terminal 42 for the respective utilized charging station 20 or the respective utilized energy accumulator is provided in every adapter 40.

A control unit 60 is arranged on the prosthesis, which identifies all electrical and/or electronic units 110, 120 in a wireless or wired manner and prompts the activation or deactivation thereof. In an alternative design, the control unit 60 can be housed in one of the units 110, 120 and, as a main control unit or master, can identify, activate, and deactivate the remaining connected components. For this purpose, all electrical and/or electronic units 110, 120 are triggered by the control unit 60, the respective provided status and functional scope is queried, and energy and/or data signals are transmitted to the respective electrical and/or electronic unit 110, 120.

A variant of the invention is shown in FIG. 2, in which instead of a bridging coupling of the two supply terminals 111, 121 via the plug connection 30 to relay energy and data and to conduct through energy and data from the charging station 20 via the respective plug 31, separate plug terminals 112, 122 are arranged on the two electrical and/or electronic units 110, 120. These plug terminals 112, 122 are provided in addition to the supply terminals 111, 121. In FIG. 2, the second supply terminal 121 is provided on the second orthopedic component 12 as an additional or optional supply terminal. The option also exists that the second supply terminal 121 can be omitted. Therefore, the second charging station 20 is only shown by dashed lines. If multiple or all orthopedic components which comprise an electrical and/or electronic unit are equipped with supply terminals, the advantage is that the charging times can be shortened, and energy and data can be transmitted individually and faster to the respective electrical and/or electronic unit.

The coupling to an external unit, i.e., to a charging station 20, can take place at multiple supply terminals 111, 121. Separately from the supply terminals 111, 121, the plug terminals 112, 122 are arranged on the respective orthopedic component 11, 12, via which a data exchange and/or energy exchange is implemented between the individual electrical and/or electronic units 110, 120. The plug 31 of the plug connection 30 can be formed differently from the plugs 21 of the charging stations 20 or occupied with other contacts, alternatively thereto, the plug terminals 112, 121 can be coupled and are compatible with the supply terminals 111, 121 and thus also with the plugs 21 of the charging stations 20. The energy supply and possibly supply with data are produced via the supply terminals 111, 121, while the connection between the electrical and/or electronic components 110, 120 is produced via the plug connection 30.

If a further component is supposed to be internally connected, a third plug 31 would be arranged on the plug connection 30, in the case of further components to be connected, further plugs.

To be able to configure and control the system made of a plurality of electrical and/or electronic components 110, 120, all electrical and/or electronic units 110, 120 which are provided in the system and coupled to one another are triggered by the control unit 60, for example, via radio, or, if the control unit 60 on an orthopedic component is connected to an electrical and/or electronic unit, via cable, wherein the other electrical and/or electronic components are preferably triggered via the plug connection 30, which can also be laid inside the orthopedic component or components. The control unit 60 queries the status of the respective unit, i.e., whether it is activated, which software status it has, what the charge level is, and which functional scope is assigned to the respective unit. Energy and/or data signals are transmitted to the respective unit on the basis of the received items of information about status and functional scope. The control unit 60 can be coupled for this purpose to the respective charging station 20, for example, wirelessly or via a wired connection. It is also possible that the control unit 60 is part of the charging station 20 or is coupled to the supply terminal 111, 121, via which energy and/or data are introduced into the system from the outside.

The control unit 60, which is arranged in the exemplary embodiment of FIG. 2 in the second orthopedic component 11 to be able to dispense with a joint-spanning cable connection, is coupled to all energy accumulators and monitors which energy level the respective energy accumulator has. The charging energy can be distributed with different priorities on the basis of the importance of the energy accumulator for the functionality of the overall system. The electrical and/or electronic units absolutely necessary for the functionality are preferably supplied with electric energy, while auxiliary units, which ensure an elevated functional scope and possibly an elevated level of comfort, are supplied with energy later or with a smaller proportion of energy. During the charging procedure, the energy distribution can be changed via the control unit 60. If the supply of external energy is terminated, the internal energy distribution between the individual energy accumulators 115, 125 within the system is controlled via the control unit 60. The energy accumulator having the presently highest charge level can thus first be assigned to a consumer, alternatively or additionally a charge exchange or energy exchange can take place between the energy accumulators to either achieve a uniform energy distribution or to supply a prioritized consumer with an elevated quantity of energy using the respective assigned energy accumulator.

Due to the items of information about the status and the type of the electrical and/or electronic component obtained by the triggering, it is possible that in the case of specific combinations of different electrical or electronic units, specific functions are not desired or have to be omitted, so that in dependence on the respective detected electrical and/or electronic unit, a corresponding function is activated or deactivated on the respective orthopedic component.

1. A system comprising:
multiple orthopedic components which are coupled to one another;
a first electronic unit, which comprises a first supply terminal, via which the first electronic unit can be supplied with at least one of energy and data from a charging station via a plug;
at least one second electronic unit arranged on one of the orthopedic components, the at least one second electrical component comprises at least one of a separate supply terminal and a plug terminal, which can be coupled to the first electronic unit to transmit at least one of energy and data via the first supply terminal or a separate plug terminal.

2. The system as claimed in claim 1, wherein the first electronic unit and the at least one second electronic unit are coupled to one another via a plug connection between the plug terminals, the supply terminals, or one plug terminal and one supply terminal.

3. The system as claimed in claim 1, wherein the supply terminals of the first electronic unit and the at least one second electronic unit are compatible with the plug terminal.

4. The system as claimed in claim 1, wherein the supply terminal of the first electronic unit is compatible with the plug terminal of the at least one second electronic unit.

5. The system as claimed in claim 1, wherein the electronic units each comprise at least one of an actuator, a control unit, a processing circuit, a sensor, a data memory, a hydraulic damper, and an energy accumulator.

6. The system as claimed in claim 1, wherein the orthopedic components are at least one of mechanically and electrically coupled to one another.

7. The system as claimed in claim 1, wherein exclusively data, exclusively electric energy, or both are transmittable via the plug terminal.

8. The system as claimed in claim 1, further comprising an adapter connectable to the first or second supply terminal or plug terminal, wherein the adapter comprises an input terminal for different charging stations.

9. The system as claimed in claim 1, wherein at least one of the supply terminal and the plug terminal comprises a fastening unit for the fixation on the respective component.

10. The system as claimed in claim 1, further comprising a control unit having multiple electronic units is coupled to accumulators for electric energy, the control unit detects charge levels of the accumulators and distributes charging current from the supply terminal to the accumulators or electric energy among the accumulators in dependence on the charge levels of the accumulators.

11. The system as claimed in claim 1, wherein the electronic units are coupled to one another at least one of electrically and to transmit data.

12. The system as claimed in claim 11, wherein all electronic units are connected to a control unit, the control unit identifies all electronic units and prompts activation or deactivation thereof.

13. A method for controlling a system as claimed in claim 1, wherein all electronic units provided in the system and coupled to one another are triggered by a control unit, the status and functional scope thereof are queried, and at least one of energy and data signals are transmitted to the respective electronic units.

14. The method as claimed in claim 13, wherein charge levels of multiple accumulators of electric energy are detected and the charging energy is distributed on the basis of the detected charge levels during a charging procedure.

15. The method as claimed in claim 13, further comprising multiple accumulators of electric energy are combined to form a composite and a supply of the electronic units from the composite is distributed centrally by the control unit.

16. The method as claimed in claim 15, wherein the accumulator of electric energy having a highest charge level is first assigned to a consumer.

17. The method as claimed in claim 13, further comprising accumulators of electric energy operable to carry out a charge exchange with one another.

18. The method as claimed in claim 13, wherein functions of the electronic units are activated or deactivated due to the coupled orthopedic components.

19. The method as claimed in claim 13, wherein the electronic units are authenticated after the coupling of the orthopedic components and at least one of a functional scope and an energy release thereof is established in dependence on the electronic units combined with one another.

20. A system comprising:
a plurality of orthopedic components coupled to one another;
a first electronic unit comprising at least one of a first supply terminal and a first plug terminal, the first electronic unit being supplied with at least one of energy and data from a charging station via the first supply terminal;
at least one second electronic unit arranged on one of the orthopedic components, the at least one second electrical component comprising at least one of a second supply terminal and a second plug terminal, the at least one second electronic unit being coupled to the first electronic unit to transmit at least one of energy and data via at least one of the first and second supply terminal or at least one of the first and second plug terminals.
